(19) United States
(12) Patent Application Publication
(10) Pub. No.: US 2005/0058730 A1
(43) Pub. Date: Mar. 17, 2005

(54) Compositions and methods for preventing or treating eyestrain

(76) Inventors: Xushan Wan, San Pedro, CA (US); Xiaozhe Liu, Alhambra, CA (US)

Correspondence Address: Xushan Wan, 647 W. 9th St, San Pedro, CA, 90731, US

(21) Appl. No.: 10/662,228

(22) Filed: Sep. 15, 2003

(51) Int. Cl. A61K 35/78

(52) U.S. Cl. 424/735; 424/764; 424/769; 424/773; 424/776; 424/777; 424/778; 424/757

(57) ABSTRACT

Compositions and methods are provided for use in eye health foods for preventing or treating eyestrain. The compositions comprise medicinal herbs or in combination medicinal herbs, vitamins, minerals, carrot root and liver of sheep. The compositions can be added to many health foods, such as cakes, candy, bread, chocolate. They are safe to the body and effective for preventing or treating eyestrain.

### BACKGROUND OF THE INVENTION

1. Field of the Invention

The present invention relates to health foods. More specifically, it relates to compositions and methods for use in eye health foods for preventing or treating eyestrain.

2. Prior Art

Information explode in present society. A main channel to obtain the information is eyes of human been. It can not do without eyes to operate computer, watch television, read books and newspaper, drive vehicle and play chess. It will lead to eyestrain and brain strain to use eyes long time or Excessively, further to a series of symptom such as eye dryness, discomfort, irritation, burning, redness, excess tearing, blurred vision, eye fatigue, soreness, strain, fear light, myopia, myopic astigmatism, or feel dizzy, giddy, headache, debility sweating, cold sweating, feel weak, or mood dejected, depressed, irritable, irascible, etc. these symptoms are named eyestrain syndrome.

How dose palliate eyestrain and dose not disturb normal studying, working and living? This is a anxious problem.

There are many methods to palliate eyestrain in our life, such as sleep, doze, rest of closing eyes, sports, music, looking far, massaging eyes, wearing sunglasses, taking vitamins, eye drops, and so on. There are other many methods to reduce irritation of eyes in the United States Patents. They are undoubted and good methods to palliate eyestrain.

Johansen et al U.S. Pat. Nos. 5,400,175 and 5,177,509 entitled “Ultraviolet radiation and Blue Light Blocking polarizing lens” discloses A lens (10) that substantially blocks horizontally polarized light and selectively blocks wavelengths between 300 and 549 nanometers. While a beneficial and calming effect is achieved by wearing only blue-blocking lenses, the addition of the polarizer (16) substantially enhances the calming effect and the improvement of vision without visual discomfort.

Elterman U.S. Pat. No. 5,502,516 entitled “Disposable/reusable sun filter” discloses A light shield is provided which includes a relatively thin but preferably rigid body of light permeable material. An arrangement is interposed between the body of light permeable material and one side of the shield directly on the lens in light intercepting relationship relative to the eye of a user of the lens.

Singhal U.S. Pat. No. 5,888,173 entitled “Health Saver Computer Break” discloses a method for providing a health break while using computers, for those aspects of health that are impacted by use of computers for sustained periods of time. These health aspects are those which cause strain and fatigue of muscles in the neck, hand and back areas. Another, important health aspect is the strain on the ciliary muscles in the eye used for focusing. The method requires the operating system to pause the application, display a health related break message and an eye related health message at different rates for preset times. This message illustrates the purpose of break and what should be done during the break. The user has the features to delay or cancel the health related break message.

Mizoguchi et al U.S. Pat. No. 5,841,466 entitled “Optical Visualizing Apparatus and Method of Controlling the same” disclose optical visualizing apparatus, when the predetermined period has passed from initiation of image display, a warning for health of eyes on the liquid crystal display means and continues image display only when the will for continuous display such as repeated input of the password is indicated, in order to give restriction to use of apparatus by children or minor persons or the like.

U.S. Pat. No. 6,261,545 disclose “Ophthalmic of neurotrophic factors, remedies for optic nerve function disorders and method for treating optic nerve function disorders.

U.S. Pat. No. 5,696,091 discloses “Use of growth factor and anti-metabolite combination to prevent or retard secondary cataract formation.

U.S. Pat. No. 5,627,208 discloses “Prostaglandin derivatives for the treatment of glaucoma or ocular hypertension.

Itoh et al U.S. Pat. No. 5,876,709 entitled “Ophthalmic Composition Containing Active Vitamin D” disclosed an ophthalmic composition for preventing corneal haze and corneal refraction anomaly observed after anterior ocular tissues are damaged or during corneal diseases comprises, as an effective component, vitamin D such as ergocalciferols and cholecalciferols or active vitamin D.

Itoh et al U.S. Pat. Nos. 6,187,331 and 6,410,006 entitled “Composition for Prophylaxis and/or Treatment of Dry Eye Syndrome Comprising Vitamin D” discloses an ophthalmic composition for prophylaxis and/or treatment of keratoconjunctivitis sicca, which is locally administered to the eyes and which comprise, as an effective component, at least one member selected from the group consisting of vitamin D, active vitamin D, and active vitamin D analogues.

Green U.S. Pat. No. 4,806,354 entitled “Health Food Composition” discloses an improved health food composition comprises: B complex vitamins, a prostaglandin E-1 precursor, a primary emulsifying agent, a flavoring agent, and a preservative.

In present years, there are some examples of treating diseases which herbal composition in the United States Patent, such as U.S. Pat. No. 6,027,728 entitled “Herbal Skin Regeneration Composition and Method” . It comprises a selection of herbal materials with curative effect combined in a powdered form for application to human skin affected with eczema, psoriasis, allergic reactions, inflammatory rash and the like.

Tao U.S. Pat. No. 6,537,581 entitled “Compositions and Methods for treating eye discomfort” discloses compositions and methods for treating eye discomfort are provided. The compositions comprise singly or in combination herbals, natural nutritional supplements, minerals and vitamins. Methods of making these compositions are also provided.

CN. Pat. No. 92114707.4 discloses the method of preparing vision health product with pig liver and liquor.

CN. Pat. No. 99114543.7 discloses the method of preparing eye health food capsule comprising five herbal powders.

CN. Pat. No. 96111591.2 discloses a herbal paste comprising many kind of herbal powders.

CN. Pat. No. 01127191.4 discloses a method and preparation of protecting eyes. It comprise many kind of herbal powders.

The above prior arts have good action to solve problem of eyes. Otherwise, the present invention is different compositions and methods from before.

No eye health food is known, however, for preventing and treating eyestrain in effective and safe heading techniques.

### SUMMARY OF THE INVENTION

The principal object of the present invention is to provide compositions and methods of eye health foods for preventing and treating eyestrain. The eye health foods have good characteristic with taking and carrying conveniently, rich nutrition, conspicuous effect.

It also is an object of the present invention to provide a basic eye health food capsule composition which comprises many kind of medicinal herbs containing active ingredients.

Another object is to provide an improved eye health food capsule with adding some vitamins and minerals.

A further object is to provide compositions and methods of eye health food such as chocolate, candy, cake, and others contain above compositions and carrot root and liver of sheep.

The foregoing objects can be accomplished by providing methods of making the eye health foods such as extracting, condensing, drying, mixing and so on.

Above research of eye health food is based on the theory of traditional Chinese medicine and new technology of modern science. The eye health food contains great quantity of natural nutrition ingredients and healthcare compositions.

The theory of traditional Chinese medicine and principle of meridians consider that the eyes is the first of nine orifices (two eyes, two ears, two nose orifices, one mouth, one anus, one vaginal orifice), the liver opens into the eyes. So liver and eyes have intimate exterior-interior relationship. “Liver Qi communicate with eyes; harmonization of the liver and therefore, the eyes can distinguish five colours. . . . five zang-organs (heart, liver, spleen, lung, kidney) do not harmonized and therefore seven orifices (two eyes, two ears, two nose orifices, one mouth) don't communicate.” The essential Qi of five zang-organs and six fu-organs ( small intestine, gallbladder, stomach, large intestine, bladder, triple-jiao energizer ) upward pour into eyes and named essences. The essences are gathered together to become eyes. The essential Qi of liver nourish black part of eye. The essential Qi of heart nourish vessel of eye. The essential Qi of lung nourish white part of eye. The essential Qi of spleen nourish eyelid. The essential Qi of kidney, liver, heart, lung and spleen merge with vessel to from ocular system. The ocular system is upward connected with brain, and out of back of neck.” (<Lingshu . Dahuolun>) “every vessel is connected with eyes.” (<Suwen. Five zang-organs grow>) “The vessel of liver meridian belongs to liver and connects with gallbladder and links ocular system, out of forehead, merges at vertex with governor vessel; its branch is into cheek from ocular system, circles lip.” (<Lingshu . Meridians and vessels>)

Bases on above quotation to know: the eyes are connected close with five zang-organs and six fu-organs, more specifically, liver is most close with eyes.

Eyestrain can lead to consumption of liver-blood because the liver has function of storing blood. The eyes excessively work and the liver transport stored blood to eyes. The eyes over consume stored liver-blood, and lead to asthenia of liver-blood.

Asthenia of liver-blood can't upward nourish eyes, therefore, will occur: eye dryness, discomfort, irritation, burning, redness, excess tearing, blurred vision, fatigue, soreness, strain, fear light, myopia, myopic astigmatism, nyctalopia and other eye diseases.

Asthenia of liver blood can't upward nourish head and brain, therefore occur: dizziness, heaviness of head, distention of head, tremor of head, headache, dull headache, migraine, scalp numbness, tinnitus cerebi pain of forehead angle, head sweating, wind-syndrome of head affecting eyes, dysphoria, insomnia and dreamful sleep, amnesia, the faculty of memory weaken.

Asthenia of liver blood will lead to disharmony between spleen and stomach. So appear: no taste for food, no appetite, indigestion, gastric upset and eructation.

Asthenia of liver blood will influence heart. Because the heart governs blood and blood circulation and mental activities. So will appear: vexation, dysphoria and palpitation, vexation and sleeplessness, susceptibility to rage due to dysphoria , flustered, nervousness and palpitation, susceptibility to fright, timidity, mental confusion, distraction, severe palpitation, precordial pain, epigastria void sensation, stifling oppression in chest, cardiac depression, anguishing sensation in heart.

The liver and the kidney share the same origin. The lumbar region is the house of the kidney. So, asthenia of liver blood will effect kidney function. Appear: dry scalp, dandruff, white hair, dry hair, trichomadesis due to hair dryness, tinnitus, tinnitus like the chirping of a cicada, deafness, aching waist and limp knees, weak legs and flaccid feet, aching pain of waist and back, lumbago, cold and heavy sensation in waist, importence with seminal emission, incomplete erection of penis, prospermia, physical fatigue and spiritual lassitude, coldness of body and limbs.

All the symptoms come from asthenia of liver blood. Eyestrain is a direct reason of producing asthenia of liver blood. Therefore, the series of symptoms or pathology produced by asthenia of liver blood and eyestrain is named eyestrain syndrome.

The key of preventing and treating eyestrain syndrome is nourishing liver blood and invigorating kidney to tranquilize mind. The present invention is designed by basis on this theory just.

Base on theory of traditional Chinese medicine, the herbal health foods have some efficiency as list below:

1. Shu di huang: Radix Rehmanniae; Root of adhesive rehmannia; [Rehmannia glutinosa (Gaertn.) Libosch.] Sweet taste and slightly warm in nature. The channel distribution of medicine is liver and kidney. The efficiency is nourishing yin and supplementing blood. It treats blurred vision.

<Zhen zhu nang> record: “powerful invigoration of blood asthenia, promoting blood circulation, nourishing Qi.”

<Ben cao gang mu> record: “replenishing bone marraw, growing muscle, promoting production of essence and blood, nourishing five zang-organs, internal injury, promoting blood circulation, benefiting ears and eyes, blacking hair and buccal carbuncle.”

<Ben cao chong xin> record: “nourishing kidney water, replenishing bone marraw, benefiting blood circulation, invigorating genuine yin, improving sight and hearing, blacking hair and buccal carbuncle.”

2. Shan zhu yu: Fructus Corni; Fruit of Asiatic cornelian cherry; (Cornus officinalis Sieb. Et Zucc.) Sour taste and slightly warm in nature. The channel distribution of medicine is liver and kidney. The efficiency is invigorating liver and kidney, stopping seminal emission Qi, consolidating collapse.

<Lei gong pao zhi lun> record: “strengthening primordial Qi, storing essence.”

<Ming yi bie lu> record: “strengthening Yin, replenishing essence, tranquilizing five zang-organs, dredging nine orifices, stopping enuresis, improving acuity of vision, strengthening power.”

3. Shan yao: Rhizoma Dioscoreae; Rhizome of common yam; (Dioscorea opposita Thunb.) Sweet taste and neutral nature. The channeled distribution of medicine is lung, spleen, and kidney. The efficiency is invigorating spleen, supplementing lung, strengthening kidney, benefiting sperm.

<Huang di Nei jing> record: “strengthening middle energizer to nourish Qi, growing muscle, improving sight and hearing in taking long.”

<Shi liao ben cao> record: “treating headache, reinforcing Yin and power.”

<Ri hua zi ben cao> record: “Replenishing five zang-organs strengthening bones and muscles, growing will and tranquilizing spirits, treating spermatorrhea and amnesia.”

4. Shi hu: Herba Dendrobii; Noble dendrobium herb; (Dendrobium nobile Lindl.) Sweet, bland and salty taste, cold in nature. The channeled distribution of medicine is stomach, lung and kidney. The efficiency is promoting fluid production to nourish stomach, clear away heat and nourish Yin.

<Ming yi bie lu> record: “replenishing essence, supplementing inernal insufficiency, soothing stomach Qi, growing muscles.”

<Yao xing lun> record: “nourishing Qi and clearing away heat, invigorating kidney to reserve essence, nourishing kidney Qi, benefiting power.”

<Ri hua zi ben cao> record: “treating consumption and weakness, strengthening bones and muscles, warming water-zang, replenishing wisdom.”

5. Wu wei zi: Fructus schisandrae; Fruit of Chinese magnolia vine; [Schisandra chinensis (Turez.)Baill.] sour taste and warm in nature. The channeled distribution of medicine is lung and kidney. The efficiency is: astringing lung, nourishing kidney, promoting fluid production, astringing sweat, stopping seminal emission.

<Ming yi bie lu> record: “nourishing five zang-organs, clearing away heat, generating Yin and promoting granulation.”

<Ri hua zi ben cao> record: “improving eye-sight, nourishing water zang-organ.”

Li Gao: “promoting fluid production to quench thirst, treating diarrhea, invigorating insufficiency of primordial Qi, astringing Qi of exhaustion, dilatation of the pupil.”

6. Ren shen: Radix Ginseng; Ginseng root; (Panax ginseng C. A. Mey.) Sweet and slightly bitter taste, warm in nature. The channeled distribution of medicine is spleen and lung. The efficiency is: invigorating primordial Qi, consolidating depletion and promoting fluid production, tranquilizing mind.

<Huang di nei jing> record: “invigorating five zang-organs, tranquilizing spirit, relieving fright, eliminating pathogenic factors, improving eye-sight, opening heart to increasing wisdom.”

<Zhen zhu nang> record: “nourishing blood, invigorating stomach Qi, purging heart-fire.”

<Yao xing lun> record: “treating insufficiency of five zang-organs Qi, five kind of overstrain and seven types of injury, consumption and weakness, invigorating five zang-organs and six fu-organs, guarding middle and defending mind.”

7. Nu zhen zi: Fructus Lucidi; Fruit of glossy privet; (Ligustrum lucidum Ait.) Bitter and sweet taste, neutral nature. The channeled distribution of medicine is liver and kidney. The efficiency is: invigorating liver and kidney, strengthening waist and knees, treating dizziness, light-headedness, tinnitus, feebleness of waist and knees, premature graying of hair.

<Ben cao gang mu> record: “strengthening Yin, invigorating waist and knees, improving eye-sight.”

<Ben cao zheng yao> record: “nourishing Yin Qi, calming Yin-fire, clearing liver-fire yet, improving eye-sight and stopping tear.”

<Huang di nei jing> record: “invigorating middle, tranquilizing five zang-organs, nourishing spirit, relieving hundreds illness.”

<Ben cao meng quan> record: “blacking hair and mustache, strengthening sinews, supplementing blood and expelling wind.”

8. Qing xiang zi: Semen Celosiae; Seed of feather cockscomb; (Celosia argentea L.) Bitter taste and cool in nature. The channeled distribution of medicine is liver. The efficiency is: expelling wind and heat, clearing liver-fire, treating reddish swollen and painful eyes, cataract, hypertension.

<Yao xing lun> record: “treating liver heat poison up to eyes, hematophthalmia, optic atrophy, nebula swelling.”

<Ri hua zi ben cao> record: “treating five zang-organs evils, replenishing brain marrow, improving sight and hearing, suppressing liver, consolidating bones and muscles.”

<Dian nan ben cao> record: “improving eye-sight, treating delacrimation, phlyctenular keratoconjunctitis.”

9. Chuan xiong: zoma Chuanxionghizome of chuanxiong; (Ligusticum chuanxiong Hort.) Pungent taste and warm in nature. The channeled distribution of medicine is liver and gallbladder. The efficiency is: promoting Qi flow to relieve depression, expelling wind and drying dampness, promoting blood circulation to arrest pain.

<Ming yi bie lu> record: “treating epiphora, over run nasal discharge and saliva.”

<Ri hua zi ben cao> record: “treating all wind, all Qi, all overstrain, all blood, invigorating five kind of overstrain, strengthening bone and muscles, regulating meridians and vessels.”

<Yi xue qi yuan> record: “supplementing blood, treating headache due to blood-asthenia.”

10. Gou qi zi: Fructus Lycii; Fruit of barbary wolfberry; (Lycium chinense Mill.) Sweet taste and neutral nature. The channeled distribution of medicine is liver and kidney. The efficiency is: nourishing kidney, moistening lung, invigorating liver, improving eye-sight, treating consumption of liver and kidney Yin, feebleness of waist and knees, dizziness, dysopia and delacrimation.

<Yao xing lun> record: “can invigorate asthenia of essence, improve eye-sight, tranquilize mind.”

<Ben cao gang mu> record: “nourishing kidney, moistening lung, improving eye-sight.”

<Ben cao lun> record: “treating liver-wind with asthenia of blood, redness and pain and itching of eyes.”“treating apoplexy dizziness, consumption.”

11. Fu ling: Poria; Indian bread; [Poria cocos (hw.) ScWolf] Sweet and bland taste, neutral nature. The channeled distribution of medicine is heart, spleen and lung. The efficiency is: promoting urination to removing dampness, invigorating spleen and harmonizing stomach, calming heart and tranquilizing mind.

<Ming yi bie lu> record: “opening chest and abdomen, regulating zang-organs Qi, suppressing kidney evil, growing Yin, invigorating Qi and power, defending middle and guarding mind.”

<Ri hua zi ben cao> record: “invigorating five kind of overstrain and seven type of injury, calming fetus, warming waist and knees, opening heart to increasing wisdom, stopping amnesia.”

<Yao xing lun> record: “promoting appetite, stopping retching tranquilizing heart and spirit.”

12. Ju hua: Flos Chrysanthemi; Flower of florists chrysanthemum; (Chrysanthemum morifolium Raimat.) Sweet and bitter taste, cool in nature. The channeled distribution of medicine is lung and liver. The efficiency is dispelling wind and clearing away heat, improving eye-sight, removing toxical material.”

<Huang di nei jing> record: “indicating all kind of diseases with dizziness and vertigo, swelling and pain, eyeball on the verge of exhaustion, lacrimation.”

<Ri hua zi ben cao> record: “promoting blood and vessel, treating dysphoria, headache, improving eye-blood.”

<Zhen zhu nang> record: “invigorating eye-blood.”

<Yong yao xin fa> record: “elimination of nebula, improving eye-sight.”

<Ben cao gang mu shi yi> record: “improving eye-sight, expelling wind, expelling liver Qi, treating dizziness, nourishing blood and moistening complexion, into blood phase.”

13. Tu si zi: Semen Cuscutae; Seed of Chinese dodder; (Cuscuta chinensis Lam.) Pungent and sweet taste, neutral nature. The channeled distribution of medicine is liver and kidney. The efficiency is invigorating liver and kidney, replenishing essence marrow, improving eye-sight.

<Huang di nei jing> record: “supplementing insufficiency, replenishing Qi and power, improving eye-sight as taking long.

<Lei gong pao zhi lun> record: “invigorating wei-Qi defenive Qi ) reinforcing tendons and vessels.”

<Ri hua zi ben cao> record: “invigorating five kind of overstrain and seven type of injury.”

<Ben cao feng yuan> record: “expelling wind and improving eye-sight, into Qi phase of liver and kidney.”

14. Mi meng hua: Flos Buddlejae; Flower of pale butterfly bush; (Buddleia officinalis Maxim.) Sweet taste and cool in nature. The channeled distribution of medicine is liver. The efficiency is: expelling wind, cooling blood, moistening liver, improving eye-sight, treating reddish swollen and painful eyes, delacrimation, photophobia, complicated cataract, marginal blepharitis.

<Kai bao ben cao> record: “indicating complicated cataract, red and dry eyes, delacrimation, treating hematophthalmia, infantile eye disease due to malnutrition.”

Liu Wan Su said: “treating photophobia.”

Wang Hao Gu said: “moistening liver dryness.”

15. Sha yuan zi: Semen Astragali Complanati; Seed of flatstem mikvetch; (Astragalus complanatus R. Br.) Sweet taste and warm in nature. The channeled distribution of medicine is liver and kidney. The efficiency is: invigorating liver, replenishing kidney, improving eye-sight, arresting nocturnal seminal emission with astringent drugs.

<Ben cao chong xin> record: “invigorating kidney, strengthening Yin, replenishing essence, improving eye-sight.”

<Ben cao qiu yuan> record: “improving eye-sight, growing muscles.”

<Ben cao gang mu> record: “invigorating kidney, treating lumbago spermatorrhea, consumption and weakness.”

<Ben cao hui yan> record: “Medicine of invigorating kidney and stopping seminal emission. Its Qi is delicate fragrance and can replenish liver and improve eye-sight, moisten pupil.”

16. Jue ming zi: Semen Cassiae; Seed of sickle senna; (Cassia tora L.) Bitter and sweet taste, cool in nature. The efficiency is: clearing liver, improving eye-sight,j promoting urination, easing bowel movement, treating red eyes due to wind-heat, complicated cataract, night blindness.

<Huang di Nei jing> record: “treating complicated cataract, conjunctivitis, red eyes pain, delacrimation; replenishing essence and sight as taking long.”

<Ri hua zi ben cao> record: “reinforcing liver Qi; replenishing essence water, better than black soy beans in pillow; treating wind-syndrome of head; improving eye-sight.”

<Hu nan Yao wu zhi> record: “improving eye-sight; promoting urination; treating dizziness.”

<Chang Yong zhong cao yao shou che> record: “clearing liver and improving eye-sight; promoting urination and easing bowel movement; treating night blindness, red eyes due to wind-heat.”

17. Hu luo bo: Radix Dauci Sativae; Carrot root; (Daucus carota L. var. sativa DC.) Sweet taste and neutral nature. The channeled distribution of medicine is lung and spleen. The efficiency is: invigorating spleen to resolve stagnation.

<Ri yong ben cao> record: “soothing the middle and descending Qi; eliminating stagnation in the stomach.”

<Ben cao gang mu> record: “descending Qi and invigorating middle; promoting chest and diaphragm, intestines and stomach; tranquilizing five zang-organs.”

<Yi lin zuan yao> record: “moistening kidney essence; invigorating primordial Yang; warming lower energrzer; expelling coldness and wetness.”

Its root contains alpha, beta carotenes, lycopene, phytofluene, vitamin B1, vitamin B2 etc.

18. Yang gan: liver of sheep; liver of Capra hircus L. and Ovis aries L. Sweet and bitter taste, cool in nature. The channeled distribution of medicine is liver. The efficiency is: invigorating blood; supplementing liver; improving eye-sight; treating dim sight due to liver-asthenia, blurred vision, night blindness, complicated cataract.

<Yao xing lun> record: “improving eye-sight to take it.”

<Qian jin . Shi zhi> record: “supplementing liver and improving eye-sight.”

<Tang ben cao> record: “treating asthenia-heat due to liver-wind, acute conjunctivitis, loss of vision.”

100 grams sheep liver contains: Protein 18.5 grams; Fat 7.2 grams; Calcium 9 mg; Phosphorous 414 mg; Iron 6.6 mg; Thiamine 0.42 mg; Folic acid 3.57 mg; Ascorbic acid 18.9 mg; Vitamin A 29900 IU.

The raw material of the eye health food is not only food, but also herbal medicine. The composition of the eye health food has great health efficiency for preventing and treating eyestrain syndrome, which is safe to the body. It has not any toxicity and side effects. It may be taken for long time. The active ingredients of the raw material do not be destroyed in the process of product. The process is rational and the methods are convenient.

### BRIEF DESCRIPTION OF THE DRAWINGS

FIG. 1 is process illustration of producing eye health food capsule HM*.

FIG. 2 is process illustration of producing eye health food capsule HV*.

FIG. 3 is process illustration of producing an additive TJ* of eye health food, such as candy or chocolate.

### DETAILED DESCRIPTION OF THE INVENTION

The present invention pertains to a composition for use in eye health food for preventing and treating eyestrain syndrome, prepared from medicinal herbs, including Shu di Huang : Radix Rehmanniae, Root of adhesive rehmannia, [Rehmannia glutinosa (Gaertn.)]; Shan zhu yu: Fructus Corni, Fruit of Asiatic cornelian cherry, (Cornus officinalis Sieb. Et Zucc.); Shan yao: Rhizome of common yam, (Dioscoyea opposita Thunb.); Shi hu: Herba Dendrobii, Noble dendrobium herb, (Dendrobium nobile Lindl.); Wu wei zi: Fructus schisandrae, Fruit of Chinese magnolia vine, [Schisandra chinensis (Turez.) Baill.]; Ren shen: Radix Ginseng, Ginseng root, (Panax ginseng C. A. Mey.); Nu zhen zi: Fructus Lucidi, Fruit of glossy privet, (Ligustrum lucidum Ait.); Qing xiang zi: Semen Celosiae, Seed of feather cockscomb, (Celosia argentea L.); Chuan xiong: Rhizome of chuanxiong, (Ligusticum chuanxiong Hort.); Gou qi zi: Fructus Lycii, Fruit of barbary wolfberry, (Lycium chinense Mill.); Fu ling: Poria, Indian bread, [Poria cocos (Schw.) Wolf]; Ju hua: Flos Chrysanthemi, Flower of florists chrysanthemum, (Chrysanthemum morifolium Raimat.); Tu si zi: Semen Cuscutae, Seed of Chinese dodder, (Cuscuta chinensis Lam.); Mi meng hua: Flos Buddlejae, Flower of pale butterfly bush, (Buddleia officinalis Maxim.); Sha yuan zi: Semen Astragali Complanati, Seed of flatstem mikvetch, (Astragalus complanatus R. Br.); Jue ming Zi: Semen Cassiae, Seed of sickle senna, (Cassia tora L.).

The formulation of composition of the present invention is below ( percentage in weight ):

| | |
|---|---|
| Shu di huang: Root of adhesive rehmannia | 11-21% |
| Shan zhu yu: Fruit of Asiatic cornelian cherry | 5-11% |
| Shan yao: Rhizome of common yam | 5-11% |
| Shi hu: Noble dendrobium herb | 5-11% |
| Wu wei zi: Fruit of Chinese magnolia vine | 5-11% |
| Ren shen: Ginseng root | 5-11% |
| Nu zhen zi: Fruit of glossy privet | 1-5% |
| Qing xiang zi: Seed of feather cockscomb | 3-7% |
| Chuan xiong: Rhizome of chuanxiong | 3-7% |
| Gou qi zi: Fruit of barbary wolfberry | 3-7% |
| Fu ling: Indian bread | 3-7% |
| Ju hua: Flower of florists chrysanthemum | 1-5% |
| Tu si zi: Seed of Chinese dodder | 3-7% |
| Mi meng hua: flower of pale butterfly bush | 1-5% |
| Sha yuan zi: Seed of flatstem mikvetch | 3-7% |
| Jue ming zi: Seed of sickle senna | 3-7% |

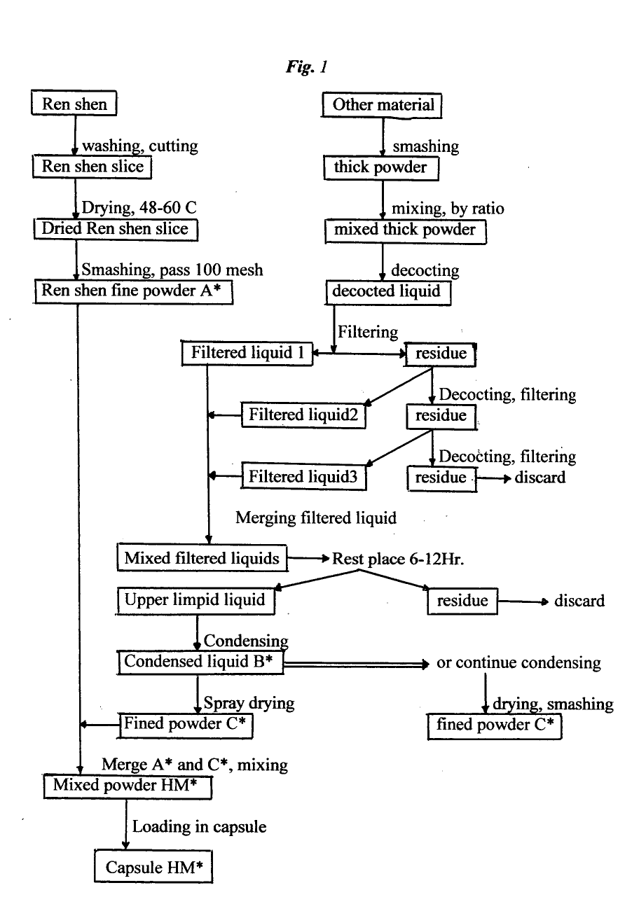

As shown in the drawings, the prepared methods of eye health food in accordance with the present invention includes steps in FIG. 1 below:

1. Wash and cut Ren shen: Ginseng root (Panax ginseng C.A. Mey.) into slices, then dry it in 48-60 degree centigrade. Smash dried Ren Shen slices and make it to pass 100 mesh. Obtain fine powder A* of Ren shen.

2. Smash other material except Ren shen into thick powder. Mix them by ratio to obtain mixed thick powder. Put water to soak the mixed thick powder about 6-12 hours. Then decoct them and keep slight boiling about 30-50 minutes. Filter the decocted liquid to obtain two parts: filtered liquid 1 and residue. Continue to decoct and filter residue as before, still obtain two parts: filtered liquid 2 and residue. Continue again to obtain filtered liquid 3 and residue. Discard residue. Merge three times filtered liquid and let it rest about 6-12 hours. Take upper limpid liquid to condense to definite concentration and obtain condensed liquid B*. discard residue.

3. Use apparatus of spraying drying. Absorb extracted condensed liquid B* to a spray head by passing through pipe and flow capacity meter. The condensed air of entering the spray head (3-6 Kg/Cm square ) increase speed by using centrifugal force via eddy current apparatus from the spray head and the condensed liquid B* be sprayed into drying room with mixing heated air current to proceed heat exchange. Collect being dried fine powder from the cloth bag and collecting pail. The mixed and dried fine powder is named medicinal powder C*.

Another method of obtaining medicinal powder C* is to keep condensing condensed liquid B* to be dry, then smash them and make them passing 100 mesh. The medicinal powder C* is obtained.

4. Merge Ren shen fine powder A* and medicinal powder C*, and then mix well. The mixed powder HM* be obtained.

5. Put the mixed powder HM* in capsules. The capsule HM* is obtained.

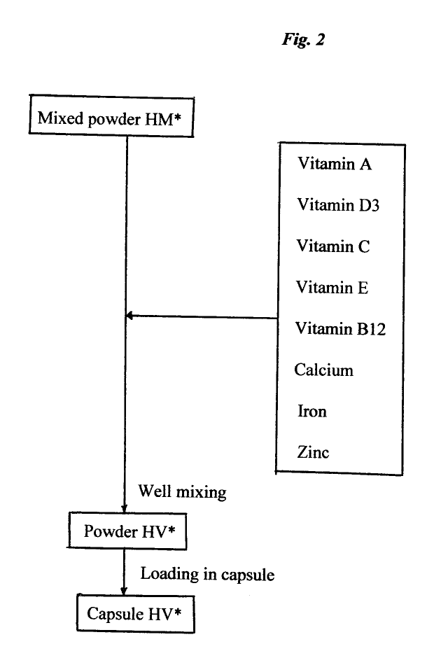

As shown in the FIG. 2, the prepared methods of eye health food capsule HV* in accordance with the present invention includes steps below:

1. Merge the mixed powder HM* and the powder of vitamins and minerals. The vitamins and minerals comprise vitamin A, vitamin D3, vitamin C, vitamin E, vitamin B12, Calcium, Iron, Zinc.

2. Mix them well. The new mixed powder HV* can be obtained.

3. Put the new mixed powder HV* in capsules. The capsules HV* can be obtained.

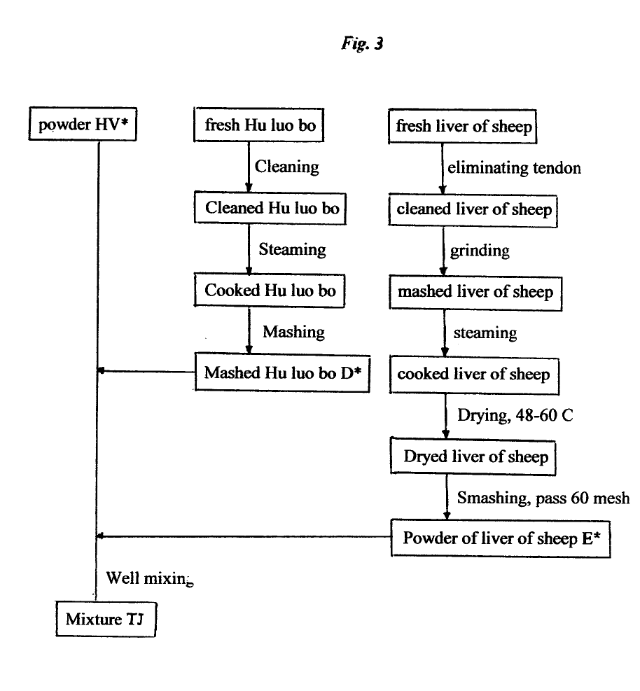

As shown in the FIG. 3, the prepared methods of eye health food additive named mixture TJ* in accordance with the present invention includes steps below:

1. Clean fresh Hu luo bo ( Carrot root ) , and steam it to be ripe, and then mash it. The mashed Carrot root D* is obtained.

2. The tendon of fresh liver of sheep (Capra hircus L.and Ovis aries L.) is eliminated, and then grind cleaned liver of sheep, steam it to be ripe, and dry it in 48-60 centigrade.

3. Smash dried liver of sheep, let it pass 60 mesh. The powder of liver of sheep E* is obtained.

4. Merge the HV*, D* and E*.

5. Mix them well. The eye health food additive mixture TJ* is obtained.

The mixture TJ* as eye health food additive can be added in candy, chocolate, cakes, bread and so on. Let these products have efficiency of preventing and treating eyestrain. They are safe to the body and effective.

The present invention is now illustrated in greater detail by way of the following examples, but it should be understood that the present invention is not to be construed as being limited thereto.

### EXAMPLE 1

Weigh Ren shen: Ginseng root (Panax ginseng C.A. Mey.) 8,000 grams, and cut it into slices, then dry it in 48-60 centigrade. Smash dried Ren shen slices and make it pass 100 mesh. The fine powder A* of Ren shen is obtained.

Weigh Shu di huang: Root of adhesive rehmannia [Rehmannia glutinosa (Gaertn.) Libosch.] 16,000 grams; Shan zhu yu: Fruit of Asiatic cornelian cherry (Cornus officinalis Sieb. Et Zucc.) 8,000 grams; Shan yao: Rhizome of common yam (Dioscorea opposita Thunb.) 8,000 grams; Shi hu: Noble dendrobium herb (Dendrobium nobile Lindl.) 8,000 grams; Wu wei zi: Fruit of Chinese magnolia vine [Schisandra chinensis (Turez.) Baill.] 8,000 grams; Qing xiang zi: Seed of feather cockscomb (Celosia argentea L.) 5,000 grams; Chuan xiong: Rhizome of chuanxiong (Ligusticum chuanxiong Hort.) 5,000 grams; Gou qi zi: Fruit of barbary wolfberry (Lycium chinense Mill.) 5,000 grams; Fu ling: Indian bread [Poria cocos (Schw.) Wolf] 5,000 grams; Tu si zi: Seed of Chinese dodder (Cuscuta chinensis Lam.) 5,000 grams; Sha yuan zi: Seed of flatstem mikvetch (Astragalus complanatus R. Br.) 5,000 grams; Jue ming zi: Seed of sickle senna (Cassia tora L.) 5,000 grams; Ju hua: Flower of florists chrysanthemum (Chrysanthemum morifolium Raimat.) 3,000 grams; Mi meng Hua: Flower of pale butterfly bush (Buddleia officinalis Maxim.) 3,000 grams; Nu zhen zi: Fruit of glossy privet (Ligustrum lucidum Ait.) 3,000 grams. Smash them into thick powder. Mix them to obtain mixed thick powder.

Put the mixed thick powder into the extractive pot and add water to drench it. Soaking it about 6-12 hours. Then decoct it and keep slight boiling about 30-50 minutes. Filter the decocted liquid to obtain two parts: filtered liquid 1 and residue. Continue to decoct and filter residue as before, still obtain two parts: filtered liquid 2 and residue. Continue again to obtain filtered liquid 3 and residue. Discard residue. Merge three times filtered liquid and let it rest about 6-12 hours. Take upper limpid liquid to condense to definite concentration and obtain condensed liquid B*. Discard residue.

Use apparatus of spraying drying. Absorb extracted condensed liquid B* to a spray head by passing through pipe and flow capacity meter. The condensed air of entering the spray head ( 3-6 Kg/Cm square ) increase speed by using centrifugal force via eddy current apparatus from the spray head and the condensed liquid B* be sprayed into drying room with mixing heated air current to proceed heat exchange. Collecting being dried fine powder from the cloth bag and collecting pail. The mixed and dried fine powder is named medicinal powder C*.

Merge Ren shen fine powder A* and medicinal powder C*, and then mix well. The mixed powder HM* is obtained.

Put the mixed powder HM* in capsules. The 40,000 capsules HM* are obtained.

Adult takes 2-3 capsules each time, 2 times each day. Child takes 1 capsule each time, and 2 times each day.

### EXAMPLE 2

After condensed liquid B* is obtained by steps of the example 1, keep condensing condensed liquid B* to be dry, then smash it and obtain medicinal powder C*.

Merge Ren shen fine powder A* and medicinal powder C*, and then mix well. The mixed powder HM* is obtained.

Put the mixed powder HM* in capsules. The capsules HM* are obtained.

### EXAMPLE 3

Merge the mixed powder HM* and the powder of vitamins and minerals. The vitamins and minerals by each capsule comprise vitamin A 1,000 IU, vitamin D3 100 IU, vitamin C 10 mg, vitamin E 8 IU, vitamin B12 3 mcg, Calcium 100 mg, Iron 1 mg, Zinc 5 mg.

Mix them well. The new mixed powder HV* can be obtained. Put the new mixed powder HV* in capsules. The capsule HV* can be obtained.

### EXAMPLE 4

Put powder HV* in candy or chocolate based on product process of preparing candy or chocolate. The quantity of 1 capsule of powder HV* makes 1-2 candy or chocolate. The candy or chocolate with powder HV* is as eye health food for preventing and treating eyestrain.

### EXAMPLE 5

Clean fresh Hu luo bo ( Carrot root ) , and steam it to be ripe, and then mash it. The mashed Carrot root D* is obtained.

The tendon of fresh liver of sheep (Capra hircus L. and Ovis aries L.) is eliminated, and then grind cleaned liver of sheep, steam it to be ripe, and dry it in 48-60 Degree centigrade.

Smash dried liver of sheep, let it pass 60 mesh. The powder of liver of sheep E* is obtained.

Merge the HV*, D* and E* in the ratio 1:3:3. Mix them well. The eye health food additive mixture TJ* is obtained.

The quantity of 1 capsule of powder HV* makes 1-2 candy or chocolate, and so on. Many kind of eye health food can be obtained by adding HV* or TJ* gradients.

1. A composition for preventing or treating eyestrain, comprises dry powdered extract from medicinal herbs, or a mixture of medicinal herbs, vitamins and minerals, or a mixture of medicinal herbs, vitamins, minerals and liver of sheep, and Carrot root.

2. The composition for preventing or treating eyestrain according to claim 1, wherein said medicinal herbs comprising 11-21 weight % of root of Adhesive rehmannia; 5-11 weight % of fruit of Asiatic cornelian cherry; 5-11 weight % of rhizome of Common yam; 5-11 weight % of Noble dendrobium herb; 5-11 weight % of fruit of Chinese magnolia vine; 5-11 weight % of Ginseng root; 1-5 weight % of fruit of Glossy privet; 3-7 weight % of seed of Feather cockscomb; 3-7 weight % of rhizome of Chuanxiong; 3-7 weight % of fruit of Barbary wolfberry; 3-7 weight % of Indian bread; 1-5 weight % of flower of Florists chrysanthemum; 3-7 weight % of seed of Chinese dodder; 1-5 weight % of flower of Pale butterfly bush; 3-7 weight % of seed of Flatstem mikvetch; 3-7 weight % of seed of sickle senna.

3. The composition for preventing or treating eyestrain according to claim 1, wherein said vitamin is a member selected from the group consisting of vitamin A, vitamin D3, vitamin C, vitamin E, vitamin B12 and a combination thereof.

4. The composition for preventing or treating eyestrain according to claim 1, wherein said mineral is a member selected from the group consisting of Calcium, Iron, Zinc and a combination thereof.

5. The composition for preventing or treating eyestrain according to claim 1, wherein said liver of sheep is dry powdered liver of Capra hircus L. or Ovis aries L. thereof.

6. The composition for preventing or treating eyestrain according to claim 1, wherein said Carrot root is ripe and mashed Carrot root.

7. The composition for preventing or treating eyestrain according to claim 1, wherein said mixture is above compositions combined in the ratio.

8. A process for producing a composition for preventing or treating eyestrain, including dry powdered extract from medicinal herbs, or a mixture of medicinal herbs, vitamins, and minerals, or a mixture of medicinal herbs, vitamins, minerals and liver of sheep, and Carrot root, said process comprising the steps of preparing them.

9. The process for producing a composition for preventing or treating eyestrain according to claim 8, Further comprising the step of preparing fine powder A* of Ginseng root, said the step including washing and cutting Ginseng root into slices, drying it in 48-60 degree centigrade, smashing dried Ginseng root slices and making it pass 100 mesh.

10. The process for producing a composition for preventing or treating eyestrain according to claim 8, further comprising the step of preparing condensed liquid B*, said the step including smashing other material except Ginseng root into thick powder, mixing them in the ratio, putting water to soak the mixed thick powder about 6-12 hours, decocting them and keeping slight boiling about 30-50 minutes, filtering the decocted liquid to obtain two parts: filtered liquid 1 and residue, continuing to decoct and filter residue two times as before, merging three times filtered liquid and letting it rest about 6-12 hours, taking upper limpid liquid to condense to definite concentration.

11. The process for producing a composition for preventing or treating eyestrain according to claim 8, further comprising the step of preparing medicinal powder C*, said the step including using apparatus of spraying drying, absorb extracted condensed liquid B* to a spray head by passing through pipe and flow capacity meter, the condensed air of entering the spray head ( 3-6 Kg/Cm square) increase speed by using centrifugal force via eddy current apparatus from the spray head and the condensed liquid B* be sprayed into drying room with mixing heated air current to proceed heat exchange, collecting dried fine powder from the cloth bag and collecting pail.

12. The process of producing a composition for preventing or treating eyestrain according to claim 8, further comprising the step of another preparing medicinal powder C*, said the step including keeping to condense condensed liquid B* to be dry, then smashing them and making them pass 100 mesh.

13. The process of producing a composition for preventing or treating eyestrain according to claim 8, further comprising the step of preparing the mixed powder HM*, said the step including merging Ginseng root fine powder A* and medicinal powder C*, mixing them well.

14. The process of producing a composition for preventing or treating eyestrain according to claim 8, further comprising the step of preparing the capsule HM*, said the step including putting the mixed powder HM* in capsules.

15. The process of producing a composition for preventing or treating eyestrain according to claim 8, further comprising the step of preparing the capsule HV*, said the step including merging the mixed powder HM* and the powder of vitamins and minerals comprising vitamin A, vitamin D3, vitamin C, vitamin E, vitamin B12, Calcium, Iron and Zinc, mixing them well, putting them in capsules.

16. The process of producing a composition for preventing or treating eyestrain according to claim 8, further comprising the step of preparing the mixture TJ* as eye health food additive, said the step including merging the HV*, D* and E*, mixing them well.

17. The process of producing a composition for preventing or treating eyestrain according to claim 16, further comprising the step of preparing the mashed Carrot root D*, said the step including cleaning fresh Carrot root, and steaming it to be ripe, and then mashing it.

18. The process of producing a composition for preventing or treating eyestrain according to claim 16, further comprising the step of preparing the powder of liver of sheep E*, said the step including eliminating tendon of fresh liver of sheep, grinding cleaned liver of sheep, steaming it to be ripe, and drying it in 48-60 degree centigrade, smashing dried liver of sheep, letting it pass 60 mesh.
